(19) United States
(12) Patent Application Publication
(10) Pub. No.: US 2006/0228755 A1
(43) Pub. Date: Oct. 12, 2006

(54) Ectopic pregnancy markers

(76) Inventors: George L. Gerton, Bryn Mawr, PA (US); Kurt T. Barnhart, Bryn Mawr, PA (US); Mary D. Sammel, Wallingford, PA (US); Jerome F. Strauss, Wyndmoor, PA (US)

Correspondence Address: PEARL COHEN ZEDEK, LLP, 1500 BROADWAY 12TH FLOOR, NEW YORK, NY, 10036, US

(21) Appl. No.: 11/314,098

(22) Filed: Dec. 22, 2005

Related U.S. Application Data
(60) Provisional application No. 60/637,774, filed on Dec. 22, 2004.

(51) Int. Cl. G01N 33/53; G01N 33/00

(52) U.S. Cl. 435/7.1; 436/86

(57) ABSTRACT

The present invention relates to methods for determining a risk or identifying a condition associated with the presence of ectopic pregnancy in a subject as well as testing a candidate compound for a therapeutic activity against an ectopic pregnancy and sorting patients based on the risk of having ectopic pregnancy. Specifically, the methods utilize novel marker proteins for assessing the risk of the patient having ectopic pregnancy.

### CROSS-REFERENCE TO RELATED APPLICATIONS

This application claims priority from U.S. Provisional Application Ser. No. 60/637,774, filed Dec. 22, 2004, which is incorporated herein by reference in its entirety.

### FIELD OF THE INVENTION

The present invention relates to methods for determining a risk or identifying a condition associated with the presence of ectopic pregnancy in a subject as well as testing a candidate compound for a therapeutic activity against an ectopic pregnancy and sorting patients based on the risk of having ectopic pregnancy. Specifically, the methods utilize novel marker proteins for assessing the risk of the patient having ectopic pregnancy.

### BACKGROUND OF THE INVENTION

A wide variety of tests have been developed for the determination of pregnancy. These methods, in general, involve the testing of blood or urine for levels of pregnancy antigens or other compounds which are indicative of pregnancy. Ectopic pregnancies, however, are not reliably distinguished from normal pregnancies by these methods, and ectopic pregnancies remain a major cause of morbidity and mortality for women.

Eectopic pregnancy occurs when an embryo implants outside of the uterus. Over 98% of ectopic pregnancies occur within the oviducts (fallopian tubes) and are commonly known as tubal pregnancies. Besides the oviducts, ectopic pregnancies can occur in other sites such as the ovary, abdominal cavity, and broad ligament. An ectopic pregnancy becomes a medical emergency when its growth causes the fallopian tube or another structure to rupture, possibly causing hemorrhage and threatening the life of the mother. Other complications resulting from ectopic pregnancy include chronic salpingitis, sterility, intestinal obstruction, and fistulae formation. Thus, it is imperative that an ectopic pregnancy be diagnosed as quickly as possible to prevent severe, possibly fatal, maternal consequences.

The present invention provides methods of diagnosing ectopic pregnancies by determining the amounts of marker proteins in a biological sample.

### SUMMARY OF THE INVENTION

In one embodiment, the invention provides a method of diagnosing or predicting of the existence of ectopic pregnancy in a subject, comprising the steps of: determining an amount of a first and a second marker in a biological sample of the subject, wherein the first marker is a protein or a peptide having a molecular mass of about 7772 Da. and said second marker being a protein or a peptide having a molecular mass of about 15,884 Da.; and comparing the amount of the first and second markers to a reference standard for the first and second markers respectively, whereby if the amount determined for either the first or the second standard falls within the range specified by the reference standard for these markers, then the subject is at a low risk of having ectopic pregnancy.

In another embodiment, the invention provides a method of diagnosing or predicting of the existence of ectopic pregnancy in a subject, comprising the steps of: determining an amount of a first and a second marker in a biological sample of said subject, said first marker being a protein or a peptide having a molecular mass of about 3962 Da. and said second marker being a protein or a peptide; and comparing the amount of said first and second marker to a reference standard for said first and second marker respectively, whereby if the amount determined for the first marker falls below the range specified for that marker by the standard for said first marker or the second standard falls outside the range defined by the reference standard for said second marker, or both, the subject is at a high risk of having ectopic pregnancy.

In one embodiment, the present invention provides a method of diagnosing or predicting of the existence of ectopic pregnancy in a subject, comprising the steps of determining an amount of a first and a second marker in a biological sample of said subject, said first marker being a peptide fragment of an alpha-fibrin protein of a biological sample of said subject having a molecular mass of about 2931 Da. and said second marker being a protein or a peptide having a molecular mass of 3962; and comparing the amount of said first and second marker to a reference standard for said first and second marker respectively, whereby if the amount determined for the alpha-fibrin peptide fragment of the biological sample exceeds the range specified for the alpha-fibrin peptide fragment of a biological sample by the standard for said alpha-fibrin protein of a biological sample or the second standard falls outside the range defined by the reference standard for said second marker, or both, the subject is at a low risk of having ectopic pregnancy.

In another embodiment, the present invention provides a method of diagnosing or predicting of the existence of ectopic pregnancy in a subject, comprising the steps of determining an amount of a first, a second, a third and a fourth marker in a biological sample of said subject, said first marker being a protein or peptide having a molecular mass of about 7772 Da.; and said third marker being a protein or a peptide having a molecular mass of 3962; and comparing the amount of said first and second marker to a reference standard for said first, second, third and fourth marker respectively, whereby if the amount determined for the first, second, third and fourth marker falls within the range specified for the first, second, third and fourth marker by the standard for said first, second, third and fourth markers respectively, the subject is at a low risk of having ectopic pregnancy.

In one embodiment, the present invention provides method for sorting a subject based on the subject being at risk of having ectopic pregnancy, comprising the steps of: obtaining a biological sample from the subject; analyzing the subject's biological sample for the amount of a first marker being a protein or a peptide having a molecular mass of about 7,772 Da, a second marker being a protein or a peptide having a molecular mass of about 15,884 Da, a third marker being a protein or a peptide having a molecular mass of about 17,717 Da and a fourth marker being a protein or a peptide having a molecular mass of about 2,941 Da.; comparing the amount of the first, second, third and fourth markers to a standard corresponding specifically to said first, second, third and fourth markers; and sorting the subjects based on the amount of the first, second, third and fourth markers relative to the range defined by the standards specific for the first, second, third and fourth markers, whereby if: the first marker falls below the threshold defined by the standard for the first marker, and the second marker falls below the threshold specified for the second marker and the third marker exceeds the threshold specified for the third marker and the fourth marker exceeds the threshold specified for the fourth marker, the subject is at a low risk of having ectopic pregnancy and is assigned a “send home” sorting criteria; or the first marker exceeds the threshold defined by the standard for the first marker, and the second marker exceeds the threshold specified for the second marker and the third marker falls below the threshold specified for the third marker and the fourth marker falls below the threshold specified for the fourth marker, the subject is at a high risk of having ectopic pregnancy and is assigned an “intervene” sorting criteria; or the amounts of the first, second, third and fourth marker are in any combination with relation to the threshold specific for the first, second, third and fourth marker, which is not covered by conditions (i) and (ii) above, the subject is assigned a “monitor” criteria, thereby being a method of sorting the subject based on the subject being at risk of having ectopic pregnancy.

### BRIEF DESCRIPTION OF THE DRAWINGS

FIG. 1 shows a histogram profiling patients participating in these studies. Most patients clustered around 6-8 weeks of gestation. Several serum samples were available from more advanced stages of normal pregnancy (light gray bars) and miscarriages (dark gray bars). Most ectopic pregnancies were diagnosed prior to 10 weeks of gestation (black bars), although there was one at 13 weeks and a number that were unknown (Un);

FIG. 2 shows a comparison of serum VEGF levels in samples from women with normal pregnancies (NP) and ectopic pregnancies (EP). Serum levels of VEGF were determined by ELISA. Values for VEGF fell into the same range for both sets of patients (20-250 pg/ml);

FIG. 3 shows a PAPP-A levels in serum samples of patients with normal pregnancies (light gray diamonds), miscarriages (dark gray triangles), and ectopic pregnancies (black squares). Serum levels of PAPP-A were determined by ELISA. In patients with a normal pregnancy, PAPP-A levels rose dramatically at around 8 weeks of gestation, whereas in women with ectopic pregnancies and miscarriages, levels remained low;

FIG. 4 shows the decision tree developed to emphasize sensitivity for patient selection. Patients with normal pregnancies (0) or ectopic pregnancies (1) were segregated based on the presence and amounts of two proteins of masses 7772 Da (M7772) and 15,884 Da (M15884). The value given by the operand “<=” represents the cut-off value used to segregate cases. The number (Cases) and percentage (%) of each patient type are given for each node, as is the total numbers of cases (N); and

FIG. 5 shows the decision tree developed to select patients by emphasizing specificity. Patients with normal pregnancies (0) or ectopic pregnancies (1) were segregated based on the presence and amounts of two proteins of masses 17717 Da (M17717) and 2941 Da (M2941). The value given by the operand “<=” represents the cut-off value used to segregate cases. The number (Cases) and percentage (%) of each patient type are given for each node, as is the total numbers of cases (N).

### DETAILED DESCRIPTION OF THE INVENTION

The present invention provides embodiments of methods for determining a risk or identifying a condition associated with the presence of ectopic pregnancy in a subject as well as testing a candidate compound for a therapeutic activity against an ectopic pregnancy and sorting patients based on the risk of having ectopic pregnancy. Specifically, the methods utilize novel marker proteins for assessing the risk of the patient having ectopic pregnancy.

In one embodiment, the invention provides a method of diagnosing or predicting the existence of ectopic pregnancy in a subject, comprising the steps of: determining an amount of a first and a second marker in a biological sample of the subject, wherein the first marker is a protein or a peptide having a molecular mass of about 7772 Da. and said second marker being a protein or a peptide having a molecular mass of about 15,884 Da.; and comparing the amount of the first and second markers to a reference standard for the first and second markers respectively, whereby if the amount determined for either the first or the second standard falls within the range specified by the reference standard for these markers, then the subject is at a low risk of having ectopic pregnancy.

In one embodiment, the methods described herein may be used for predicting the probability of the subject developing, or being at risk of developing, or having developed ectopic pregnancy. In one embodiment, the methods of described herein may be used for determining a risk or identifying a condition associated with the presence of abnormal pregnancy, such as ectopic pregnancy in one embodiment, or miscarriage or hydatiform pregnancy in other embodiments. In one embodiment, the methods described herein, are designed to be employed at the point of care, such as in emergency rooms and operating rooms, or in any situation in which a rapid and accurate result is desired.

In one embodiment, the biological sample utilized in method of the present invention is a serum sample. In another embodiment, the biological sample is a blood plasma sample. In another embodiment, the biological sample is a vaginal fluid sample. In another embodiment, the biological sample is a urine sample. In another embodiment, the biological sample is a saliva sample. In one embodiment, the marker protein or peptides used for the methods of the invention are obtained from a biological sample given by the subject. The sample to be analyzed may consist in one embodiment of, or comprise blood, sera, urine, mucosa, feces, epidermal sample, skin sample, cheek swab, sperm, amniotic fluid, cultured cells, bone marrow sample or chorionic villi, and the like.

In one embodiment, the second marker protein or peptide has a molecular mass of about 15884 Da. A combination of an abnormally low level of a marker protein with a molecular mass of about 7772 Da. and an abnormally high level of a marker protein with a molecular mass of about 15884 Da. was shown in one embodiment of the present invention to predict ectopic pregnancies with over 98% sensitivity (Example 1). Thus, in one embodiment, subjects that do not exhibit this combination (i.e. subjects that have either a normal level of (a) the 15884 Da. protein; or (b) the 7772 dalton protein; or (c) both) have a high likelihood of not having an ectopic pregnancy. In one embodiment, subject exhibiting normal level of the 15,884 Da. protein or peptide and normal levels of the 7,772 protein or peptide, once diagnosed according to the methods described herein, are assigned a “send home” criteria for diagnosis purposes.

In another embodiment, the second marker protein or peptide is any other marker protein or peptide that can be detected by a detection method of the embodiments of methods described herein, and wherein each possibility represents a separate embodiment of the present invention.

In another embodiment, the invention provides a method of diagnosing or predicting of the existence of ectopic pregnancy in a subject, comprising the steps of: determining an amount of a first and a second marker in a biological sample of said subject, said first marker being a protein or a peptide having a molecular mass of about 3962 Da. and said second marker being a protein or a peptide; and comparing the amount of said first and second marker to a reference standard for said first and second marker respectively, whereby if the amount determined for the first marker falls below the range specified for that marker by the standard for said first marker or the second standard falls outside the range defined by the reference standard for said second marker, or both, the subject is at a high risk of having ectopic pregnancy.

In another embodiment, the present invention provides a method of diagnosing a female subject with a suspected ectopic pregnancy, comprising (a) determining an amount of a first marker protein or peptide of a biological sample of the female subject, the first marker protein or peptide having a molecular mass of about 15884 Da.; (b) comparing the amount of a first marker protein or peptide to a reference standard for the first marker protein or peptide; (c) determining an amount of a second marker protein or peptide of the biological sample or an additional biological sample of the female subject; and (d) comparing the amount of a second marker protein or peptide to a reference standard for the second marker protein or peptide. A diagnosis is made based on the amounts or levels of each marker protein, compared with its respective reference standard. By this method, if either (i) the amount of a first marker protein or peptide falls within a range defined by the reference standard for the first marker protein or peptide; or if (ii) the amount of a second marker protein or peptide falls within a range defined by the reference standard for the second marker protein or peptide; or if both (i) and (ii) are correct, then the female subject is at a low risk of having an ectopic pregnancy.

In one embodiment, the second marker protein or peptide has a molecular mass of about 7772 Da. In another embodiment, the second marker protein or peptide is any other marker protein or peptide that can be detected by a detection method of the present invention. Each possibility represents a separate embodiment of the present invention.

In one embodiment, the present invention provides a method of diagnosing or predicting of the existence of ectopic pregnancy in a subject, comprising the steps of determining an amount of a first and a second marker in a biological sample of said subject, said first marker being of an alpha-fibrin peptide fragment of a biological sample of said subject having a molecular mass of about 2931 Da and said second marker being a protein or a peptide having a molecular mass of 3962; and comparing the amount of said first and second marker to a reference standard for said first and second marker respectively, whereby if the amount determined for the alpha-fibrin peptide fragment of the biological sample exceeds the range specified for the alpha-fibrin peptide fragment of a biological sample by the standard for said alpha-fibrin peptide fragment of a biological sample, or the second standard falls outside the range defined by the reference standard for said second marker, or both, the subject is at a low risk of having ectopic pregnancy.

In another embodiment, the present invention provides a method of diagnosing a female subject with a suspected ectopic pregnancy, comprising (a) determining an amount of a first marker protein or peptide of a biological sample of the female subject, the first marker protein or peptide having a molecular mass of about 3962 Da.; (b) comparing the amount of a first marker protein or peptide to a reference standard for the first marker protein or peptide; (c) determining an amount of a second marker protein or peptide of the biological sample or an additional biological sample of the female subject; and (d) comparing the amount of a second marker protein or peptide to a reference standard for the second marker protein or peptide. A diagnosis is made based on the amounts or levels of each marker protein, compared with its respective reference standard. By this method, if either (i) the amount of a first marker protein or peptide is smaller than a lower limit of a range defined by the reference standard for the first marker protein or peptide; or if (ii) the amount of a second marker protein or peptide falls outside a range defined by the reference standard for the second marker protein or peptide; or if both (i) and (ii) are correct, then the female subject is at a high risk of having an ectopic pregnancy.

In one embodiment, the second marker protein or peptide is a fragment of an alpha-fibrin peptide fragment. A combination of a normal level of a marker protein with a molecular mass of about 3962 Da. and a normal level of a marker protein with a molecular mass of about 2941 (found to be a fragment of alpha-fibrin protein; Example 3) was shown in the present invention to predict ectopic pregnancies with high specificity (Example 2). Thus, in this embodiment, subjects that do not exhibit this combination (i.e. subjects that have (a) an abnormally low normal level of the 3962 dalton protein; or (b) an abnormally high level of the fragment of alpha-fibrin protein; or (c) both (a) and (b) have a high likelihood of having an ectopic pregnancy.

In one embodiment, the second marker protein or peptide has a molecular mass of about 17717 Da. An abnormally low level of this peak was shown to correlated with ectopic pregnancies (Example 1). In another embodiment, the second marker protein or peptide is any other marker protein or peptide that can be detected by a detection method of the present invention. Each possibility represents a separate embodiment of the present invention.

In another embodiment, the present invention provides a method of diagnosing a female subject with a suspected ectopic pregnancy, comprising (a) determining an amount of a fragment of an alpha-fibrin protein of a biological sample of the female subject; (b) comparing the amount of a fragment of an alpha-fibrin protein to a reference standard for the fragment of an alpha-fibrin protein; (c) determining an amount of a second marker protein or peptide of the biological sample or an additional biological sample of the female subject; and (d) comparing the amount of a second marker protein or peptide to a reference standard for the second marker protein or peptide. A diagnosis is made based on the amounts or levels of each marker protein, compared with its respective reference standard. By this method, if either (i) the amount of a fragment of an alpha-fibrin protein is greater than an upper limit of a range defined by the reference standard for the fragment of an alpha-fibrin protein; or if (ii) the amount of a second marker protein or peptide falls outside a range defined by the reference standard for the second marker protein or peptide; or if both (i) and (ii) are correct, then the female subject is at a high risk of having an ectopic pregnancy.

In one embodiment, the second marker protein or peptide has a molecular mass of about 3962 Da. A combination of a normal level of a marker protein with a molecular mass of about 3962 Da. and a fragment of alpha-fibrin protein was shown in the present invention to predict non-ectopic pregnancies with high specificity, as described above. In another embodiment, the second marker protein or peptide is any other marker protein or peptide that can be detected by a detection method of the present invention. Each possibility represents a separate embodiment of the present invention

In another embodiment, the second marker protein or peptide has a molecular mass of about 17717 Da. A combination of a normal level of a marker protein with a molecular mass of about 17717 Da. and a fragment of alpha-fibrin protein was shown in the present invention to predict non-ectopic pregnancies with a specificity of over 93% (Example 1). Thus, in this embodiment, subjects that do not exhibit this combination (i.e. subjects that have (a) an abnormally low normal level of the 17717 dalton protein; or (b) an abnormally high level of the fragment of alpha-fibrin protein; or (c) both (a) and (b) have a high likelihood of having an ectopic pregnancy.

In another embodiment, the second marker protein or peptide is any other marker protein or peptide that can be detected by a detection method of the present invention. Each possibility represents a separate embodiment of the present invention.

In another embodiment, the present invention provides a method of diagnosing or predicting of the existence of ectopic pregnancy in a subject, comprising the steps of determining an amount of a first, a second, a third and a fourth marker in a biological sample of said subject, said first marker being a protein or peptide having a molecular mass of about 7772 Da.; and said third marker being a protein or a peptide having a molecular mass of 3962; and comparing the amount of said first and second marker to a reference standard for said first, second, third and fourth marker respectively, whereby if the amount determined for the first, second, third and fourth marker falls within the range specified for the first, second, third and fourth marker by the standard for said first, second, third and fourth markers respectively, the subject is at a low risk of having ectopic pregnancy.

In another embodiment, described in Examples 1-2, the present invention provides a method of diagnosing a female subject with a suspected ectopic pregnancy, comprising (a) determining an amount of a first marker protein or peptide of a biological sample of the female subject, the first marker protein or peptide having a molecular mass of about 7772 Da.; (b) comparing the amount of a first marker protein or peptide to a reference standard for the first marker protein or peptide; (c) determining an amount of a second marker protein or peptide of the biological sample of step (a) or an additional biological sample of the female subject; (d) comparing the amount of a second marker protein or peptide to a reference standard for the second marker protein or peptide; (e) determining an amount of a third marker protein or peptide of the biological sample of step (a) or an additional biological sample of the female subject, the third marker protein or peptide having a molecular mass of about 3962 Da.; (f) comparing the amount of a third marker protein or peptide to a reference standard for the third marker protein or peptide; (g) determining an amount of a fourth marker protein or peptide of the biological sample of step (a) or an additional biological sample of the female subject; and (h) comparing the amount of a fourth marker protein or peptide to a reference standard for the fourth marker protein or peptide. This method combines above-methods of the present invention for assessing a low risk of having an ectopic pregnancy, and methods of the present invention for assessing a high risk of having an ectopic pregnancy. The diagnosis is made based on the outcome of assessing the levels of the four proteins, as will now be described:

Attainment of outcomes (i) and (ii) indicates that the female subject is at a low risk of having an ectopic pregnancy: Outcome (i) is defined as either (i-a) the amount of a first marker protein or peptide falls within a range defined by the reference standard for the first marker protein or peptide; or (i-b) the amount of a second marker protein or peptide falls within a range defined by the reference standard for the second marker protein or peptide; or (i-c) both (i-a) and (i-b) are correct. Outcome (ii) is defined as both (ii-a) the amount of a third marker protein or peptide falls within a range defined by the reference standard for the third marker protein or peptide; and (ii-b) the amount of a fourth marker protein or peptide falls within a range defined by the reference standard for the fourth marker protein or peptide.

Attainment of outcomes (iii) and (iv) indicates that the female subject is at a high risk of having an ectopic pregnancy: Outcome (iii) is defined as: both (iii-a) the amount of a first marker protein or peptide is smaller than a lower limit of a range defined by the reference standard for the first marker protein or peptide; and (iii-b) the amount of a second marker protein or peptide falls outside a range defined by the reference standard for the second marker protein or peptide. Outcome (iv) is defined as either (iv-a) the amount of a third marker protein or peptide is smaller than a lower limit of a range defined by the reference standard for the third marker protein or peptide; or (iv-b) the amount of a fourth marker protein or peptide falls outside a range defined by the reference standard for the fourth marker protein or peptide; or (iv-c) both (iv-a) and (iv-b) are correct.

In one embodiment, if neither (I) both outcomes (i) and (ii) are observed, or (II) both outcomes (iii) and (iv) are observed, then the status of the subject has not been determined by this method.

In one embodiment, the second marker protein or peptide has a molecular mass of about 15884 Da. In another embodiment, the second marker protein or peptide is any other marker protein or peptide that can be detected by a detection method of the present invention. Each possibility represents a separate embodiment of the present invention.

In one embodiment, the fourth marker protein or peptide is a peptide fragment of an alpha-fibrin protein. In another embodiment, the fourth marker protein or peptide is any other marker protein or peptide that can be detected by a detection method of the present invention. Each possibility represents a separate embodiment of the present invention.

In another embodiment, the present invention provides a method of diagnosing a female subject with a suspected ectopic pregnancy, comprising (a) determining an amount of a first marker protein or peptide of a biological sample of the female subject, the first marker protein or peptide having a molecular mass of about 15884 Da.; (b) comparing the amount of a first marker protein or peptide to a reference standard for the first marker protein or peptide; (c) determining an amount of a second marker protein or peptide of the biological sample of step (a) or an additional biological sample of the female subject; (d) comparing the amount of a second marker protein or peptide to a reference standard for the second marker protein or peptide; (e) determining an amount of a third marker protein or peptide of the biological sample of step (a) or an additional biological sample of the female subject, the third marker protein or peptide having a molecular mass of about 3962 Da.; (f) comparing the amount of a third marker protein or peptide to a reference standard for the third marker protein or peptide; (g) determining an amount of a fourth marker protein or peptide of the biological sample of step (a) or an additional biological sample of the female subject; and (h) comparing the amount of a fourth marker protein or peptide to a reference standard for the fourth marker protein or peptide. This method is similar to the preceding method, except that the protein corresponding to the 15884 dalton peak is used as the first marker protein, in place of the protein corresponding to the 7772 dalton peak. Thus, the diagnosis is made in a manner similar to the preceding method. In this case, however, it is an abnormally high (not low) concentration of the first marker protein that indicates an ectopic pregnancy.

In another embodiment, the second marker protein or peptide has a molecular mass of about 7772 Da. In another embodiment, the second marker protein or peptide is any other marker protein or peptide that can be detected by a detection method of the present invention. Each possibility represents a separate embodiment of the present invention.

In one embodiment, the fourth marker protein or peptide is a fragment of an alpha-fibrin protein. In another embodiment, the fourth marker protein or peptide is any other marker protein or peptide that can be detected by a detection method of the present invention. Each possibility represents a separate embodiment of the present invention.

In another embodiment, the present invention provides a method of diagnosing a female subject with a suspected ectopic pregnancy, comprising (a) determining an amount of a first marker protein or peptide of a biological sample of the female subject, the first marker protein or peptide having a molecular mass of about 7772 Da.; (b) comparing the amount of a first marker protein or peptide to a reference standard for the first marker protein or peptide; (c) determining an amount of a second marker protein or peptide of the biological sample of step (a) or an additional biological sample of the female subject; (d) comparing the amount of a second marker protein or peptide to a reference standard for the second marker protein or peptide; (e) determining an amount of a fragment of an alpha-fibrin protein of the biological sample of step (a) or an additional biological sample of the female subject, the third marker protein or peptide having a molecular mass of about 3962 Da.; (f) comparing the amount of a fragment of an alpha-fibrin protein to a reference standard for the fragment of an alpha-fibrin protein; (g) determining an amount of a fourth marker protein or peptide of the biological sample of step (a) or an additional biological sample of the female subject; and (h) comparing the amount of a fourth marker protein or peptide to a reference standard for the fourth marker protein or peptide. The diagnosis is made based on the outcome of assessing the levels of the four proteins, as will now be described:

Attainment of outcomes (i) and (ii) indicates that the female subject is at a low risk of having an ectopic pregnancy: Outcome (i) is defined as either (i-a) the amount of a first marker protein or peptide falls within a range defined by the reference standard for the first marker protein or peptide; or (i-b) the amount of a second marker protein or peptide falls within a range defined by the reference standard for the second marker protein or peptide; or (i-c) both (i-a) and (i-b) are correct. Outcome (ii) is defined as both (ii-a) the amount of a fragment of an alpha-fibrin protein falls within a range defined by the reference standard for the third marker protein or peptide; and (ii-b) the amount of a fourth marker protein or peptide falls within a range defined by the reference standard for the fourth marker protein or peptide.

Attainment of outcomes (iii) and (iv) indicates that the female subject is at a high risk of having an ectopic pregnancy: Outcome (iii) is defined as: both (iii-a) the amount of a first marker protein or peptide is smaller than a lower limit of a range defined by the reference standard for the first marker protein or peptide; and (iii-b) the amount of a second marker protein or peptide falls outside a range defined by the reference standard for the second marker protein or peptide. Outcome (iv) is defined as either (iv-a) the amount of a fragment of an alpha-fibrin protein is greater than an upper limit of a range defined by the reference standard for the third marker protein or peptide; or (iv-b) the amount of a fourth marker protein or peptide falls outside a range defined by the reference standard for the fourth marker protein or peptide; or (iv-c) both (iv-a) and (iv-b) are correct.

In another embodiment, the second marker protein or peptide has a molecular mass of about 15884 Da. In another embodiment, the second marker protein or peptide is any other marker protein or peptide that can be detected by a detection method of the present invention. Each possibility represents a separate embodiment of the present invention.

In another embodiment, the fourth marker protein or peptide has a molecular mass of about 3962 Da. In another embodiment, the fourth marker protein or peptide has a molecular mass of about 17717 Da. In another embodiment, the fourth marker protein or peptide is any other marker protein or peptide that can be detected by a detection method of the present invention. Each possibility represents a separate embodiment of the present invention.

In another embodiment, the present invention provides a method of diagnosing a female subject with a suspected ectopic pregnancy, comprising (a) determining an amount of a first marker protein or peptide of a biological sample of the female subject, the first marker protein or peptide having a molecular mass of about 15884 Da.; (b) comparing the amount of a first marker protein or peptide to a reference standard for the first marker protein or peptide; (c) determining an amount of a second marker protein or peptide of the biological sample of step (a) or an additional biological sample of the female subject; (d) comparing the amount of a second marker protein or peptide to a reference standard for the second marker protein or peptide; (e) determining an amount of a fragment of an alpha-fibrin protein; (f) comparing the amount of a fragment of an alpha-fibrin protein to a reference standard for the third marker protein or peptide; (g) determining an amount of a fourth marker protein or peptide of the biological sample of step (a) or an additional biological sample of the female subject; and (h) comparing the amount of a fourth marker protein or peptide to a reference standard for the fourth marker protein or peptide. This method is similar to the preceding method utilizing the protein corresponding to the 7772 Da peak and the fragment of an alpha-fibrin protein, except that the protein corresponding to the 15884 dalton peak is used as the first marker protein, in place of the protein corresponding to the 7772 dalton peak. Thus, the diagnosis is made in a manner similar to the preceding method. In this case, however, it is an abnormally high (not low) concentration of the first marker protein that indicates an ectopic pregnancy.

In one embodiment, the second marker protein or peptide has a molecular mass of about 7772 Da. In another embodiment, the second marker protein or peptide is any other marker protein or peptide that can be detected by a detection method of the present invention. Each possibility represents a separate embodiment of the present invention.

In another embodiment, the fourth marker protein or peptide has a molecular mass of about 3962 Da. In another embodiment, the fourth marker protein or peptide has a molecular mass of about 17717 Da. In another embodiment, the fourth marker protein or peptide is any other marker protein or peptide that can be detected by a detection method of the present invention. Each possibility represents a separate embodiment of the present invention.

In one embodiment, a diagnostic method of the present invention is used in conjunction with a pregnancy test. In one embodiment, the pregnancy test is a hCG test. In another embodiment, the pregnancy test is any other pregnancy test known in the art. In another embodiment, the diagnostic method is used independently of a pregnancy test. Each possibility represents a separate embodiment of the present invention.

In another embodiment, the present invention provides a method of testing a candidate compound for a therapeutic activity against an ectopic pregnancy, comprising (a) diagnosing an initial clinical state of a female subject by a diagnostic method of the present invention; (b) contacting the female subject with the candidate compound; and (c) diagnosing a subsequent clinical state of the female subject by the diagnostic method, wherein the diagnosing a subsequent clinical state is performed after step (b), whereby a diagnosis of the ectopic pregnancy in step (a) and a diagnosis other than the ectopic pregnancy in step (c) indicates that the candidate compound has a therapeutic activity against an ectopic pregnancy.

Each diagnostic method of the present invention can be used in the above method of testing a candidate compound for a therapeutic activity against an ectopic pregnancy, and each diagnostic method represents a separate embodiment of the present invention.

In another embodiment, the present invention provides a method of testing a candidate compound for a therapeutic activity against an ectopic pregnancy, comprising (a) diagnosing a clinical state of a first female subject by a diagnostic method of the present invention, wherein the first female subject has not been contacted with the candidate compound; (b) diagnosing a clinical state of a second female subject by the a diagnostic method of the present invention, wherein the second female subject has not been contacted with the candidate compound; and (c) comparing the clinical state of a first female subject to the clinical state of a second female subject, whereby a decreased incidence of the ectopic pregnancy in the first female subject relative to the second female subject indicates that the candidate compound has a therapeutic activity against an ectopic pregnancy.

Each diagnostic method of the present invention can be used in the above method of testing a candidate compound for a therapeutic activity against an ectopic pregnancy, and each diagnostic method represents a separate embodiment of the present invention.

In another embodiment, the present invention provides a method of testing a candidate ectopic pregnancy marker, comprising (a) determining an amount of the candidate ectopic pregnancy marker in a biological sample from a female subject at risk of having an ectopic pregnancy; (b) obtaining a pregnancy status of the female subject, wherein the pregnancy status is selected from: intra-uterine pregnancy, ectopic pregnancy, and miscarriage; (c) repeating steps (a)-(b) for a population of female subjects at risk of having an ectopic pregnancy; and (d) ascertaining whether a correlation exists between the amount and the pregnancy status, wherein a presence of the correlation indicates that the candidate ectopic pregnancy marker is useful in diagnosing an ectopic pregnancy.

In another embodiment, the ascertaining utilizes a classification and regression tree analysis. CART analysis was used in Examples of the present invention to identify protein peaks that correlate with ectopic and non-ectopic pregnancy diagnoses, and can be similarly used for any other indicator of the status of a subject. Use of CART analysis is well known in the art, and is described, for example, in Vlahou A et al, J Biomed Biotechnol 2003; 2003(5):308-314

In another embodiment, the ascertaining is performed by any other statistical method known in the art. Each statistical method represents a separate embodiment of the present invention.

In another embodiment, the present invention provides a method of testing a candidate ectopic pregnancy marker, comprising (a) determining an amount of the candidate ectopic pregnancy marker in a biological sample from a female subject at risk of having an ectopic pregnancy; (b) obtaining a clinical factor of the female subject, wherein the clinical factor is selected from: a maternal age, a gestational age, a clinical history, and a serum human chorionic gonadotropin (hCG) level; (c) obtaining a pregnancy status of the female subject, wherein the pregnancy status is selected from: intrauterine pregnancy, ectopic pregnancy, and miscarriage; (d) repeating steps (a)-(c) for a population of female subjects at risk of having an ectopic pregnancy; and (e) ascertaining whether a correlation exists between (i) a mathematical function of the concentration and the clinical factor; and (ii) the pregnancy status, wherein a presence of the correlation indicates that the candidate ectopic pregnancy marker is useful in diagnosing an ectopic pregnancy.

In one embodiment, the present invention provides method for sorting a subject based on the subject being at risk of having ectopic pregnancy, comprising the steps of: obtaining a biological sample from the subject; analyzing the subject's biological sample for the amount of a first marker being a protein or a peptide having a molecular mass of about 7,772 Da, a second marker being a protein or a peptide having a molecular mass of about 15,884 Da, a third marker being a protein or a peptide having a molecular mass of about 17,717 Da and a fourth marker being a protein or a peptide having a molecular mass of about 2,941 Da.; comparing the amount of the first, second, third and fourth markers to a standard corresponding specifically to said first, second, third and fourth markers; and sorting the subjects based on the amount of the first, second, third and fourth markers relative to the range defined by the standards specific for the first, second, third and fourth markers, whereby if: the first marker falls below the threshold defined by the standard for the first marker, and the second marker falls below the threshold specified for the second marker and the third marker exceeds the threshold specified for the third marker and the fourth marker exceeds the threshold specified for the fourth marker, the subject is at a low risk of having ectopic pregnancy and is assigned a “send home” sorting criteria; or the first marker exceeds the threshold defined by the standard for the first marker, and the second marker exceeds the threshold specified for the second marker and the third marker falls below the threshold specified for the third marker and the fourth marker falls below the threshold specified for the fourth marker, the subject is at a high risk of having ectopic pregnancy and is assigned an “intervene” sorting criteria; or the amounts of the first, second, third and fourth marker are in any combination with relation to the threshold specific for the first, second, third and fourth marker, which is not covered by conditions (i) and (ii) above, the subject is assigned a “monitor” criteria, thereby being a method of sorting the subject based on the subject being at risk of having ectopic pregnancy.

Using these methods, each of the peaks identified in the present invention will be sequenced to identify the protein comprising them, as described in Example 4. Immunological or other assays will then be developed for detection of each of these proteins, further improving the assays of the present invention.

Methods of protein sequencing are well known in the art, and are described, for example, in Lodish et al, Molecular Cell Biology, Fourth Edition, W. H. FREEMAN, 2000; and Berg et al, Biochemistry, Fifth Edition, 2002). Each protein sequencing method represents a separate embodiment of the present invention.

In one embodiment, the fragment of an alpha-fibrin protein of the present invention has a molecular mass of about 2931. In another embodiment, the fragment of an alpha-fibrin protein comprises a peptide having a sequence set forth in SEQ ID No: 1. In another embodiment, the fragment of an alpha-fibrin protein comprises a sequence homologous to SEQ ID No: 1. In another embodiment the fragment of an alpha-fibrin protein comprises a sequence that is a variant of SEQ ID No: 1. In another embodiment, the fragment of an alpha-fibrin protein consists of SEQ ID No: 1. In another embodiment, the fragment of an alpha-fibrin protein is a homologue of SEQ ID No: 1. In another embodiment, the fragment of an alpha-fibrin protein is a variant of SEQ ID No: 1 (Example 3). Each possibility represents a separate embodiment of the present invention.

The term “about” refers in one embodiment to quantitative terms plus or minus 5%, or in another embodiment plus or minus 10%, or in another embodiment plus or minus 15%, or in another embodiment plus or minus 20%.

The terms “homology,” “homologous,” etc, when in reference to any protein or peptide, refer in one embodiment, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

In another embodiment, the term “homology,” when in reference to any nucleic acid sequence similarly indicates a percentage of nucleotides in a candidate sequence that are identical with the nucleotides of a corresponding native nucleic acid sequence.

Homology is, in one embodiment, determined in the latter case by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, homology is determined is via determination of candidate sequence hybridization, methods of which are well described in the art (See, for example, “Nucleic Acid Hybridization” Hames, B. D., and Higgins S. J., Eds. (1985); Sambrook et al., 1989, Molecular Cloning, A Laboratory Manual, (Volumes 1-3) Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the present invention.

In one embodiment, the method of determining the amount of one or more of the proteins or peptides of the present invention comprises an immunological assay. In one embodiment, the immunological assay is a radio-immunoassay (RIA). In another embodiment, the immunological assay is an enzyme-linked immunosorbent assay (ELISA). In another embodiment, the immunological assay is a sandwich immunoassay. In another embodiment, the immunological assay is any other immunological assay known in the art. Each immunological assay represents a separate embodiment of the present invention.

Methods of performing immunological assays are well known in the art, and are described, for example, in Current Protocols in Immunology, John Wiley & Sons, 2004. Each immunological assay represents a separate embodiment of the present invention.

In another embodiment, the method of determining the amount of one or more of the proteins or peptides of the present invention comprises a surface-enhanced laser desorption/ionization (SELDI) assay.

In one embodiment, the SELDI utilizes a weak cation exchange (WCX2) chemistry. In another embodiment, the SELDI utilizes Immobilized Metal Affinity Capture (IMAC) chemistry. In one embodiment, the IMAC chemistry comprises a copper ion. In another embodiment, the chemistry is similar to WCX2 chemistry (e.g. an improved or altered version thereof). In another embodiment, the chemistry is similar to IMAC chemistry. Each possibility represents a separate embodiment of the present invention.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects. In one embodiment, the term “subject” for purposes of diagnosis, or risk factors assessment refers to a human subject who is pregnant. The subject, in one embodiment is at risk of or exhibiting symptoms associated with ectopic pregnancy due to having low serum concentrations of PAPP-C, and the like.

In one embodiment, determination of the marker protein or peptide used in embodiments of methods described herein is done by using an immunological assay, a surface-enhanced laser desorption/ionization (SELDI) assay, a mass spectrometry, or a combination thereof.

The following examples are presented in order to more fully illustrate the preferred embodiments of the invention. They should in no way be construed, however, as limiting the broad scope of the invention.

### EXPERIMENTAL DETAILS SECTION

### Example 1

### Identification of Four Useful Ectopic Pregnancy Markers

### Materials and methods

Patients and Serum Samples

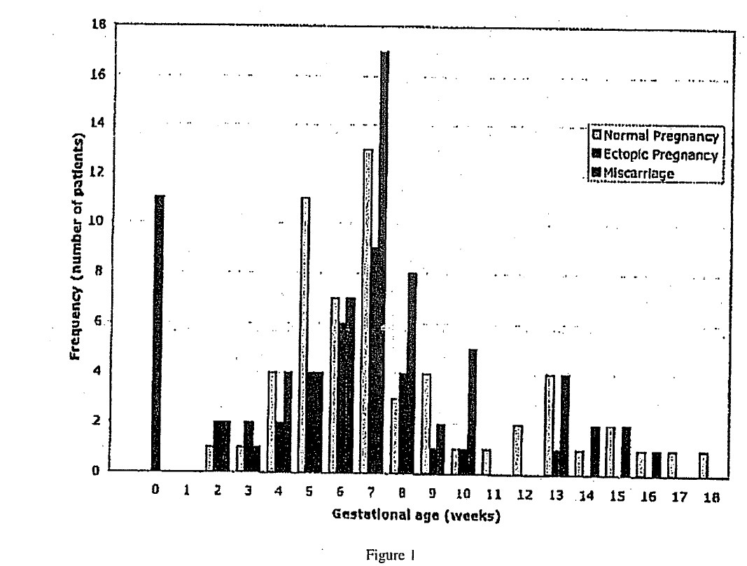

The subjects for these studies were women that presented at the Hospital of the University of Pennsylvania with suspicion of an ectopic pregnancy (pelvic pain and/or bleeding in the first trimester of pregnancy). The subjects included patients who had normal intra-uterine pregnancies, ectopic pregnancies, and spontaneous abortions (miscarriages) (FIG. 1). The approximate racial breakdown of the subjects was as follows: African American, 36%; Caucasian, 3%; Asian, 2%; Hispanic, 1%; unknown, 58%. Serum samples from 139 women (62 with an ectopic pregnancy and 77 with a normal intrauterine pregnancy) were used in this study. The gestational ages ranged from 2-18 weeks. Most patients with ectopic pregnancy were below 9 weeks of gestational age; gestational age was unclassified in 11 patients. The acquisition and use of the serum samples was approved by the Institutional Review Board of the University of Pennsylvania.

Serum Collection Process

Serum samples were obtained directly from the clinical pathology laboratory. The collection process was standard clinical procedure for blood drawing, coagulation, or storage. Once obtained from the clinical laboratory, samples were divided into small (20 microliter (μL)) aliquots and stored at −80° C. Aliquots were thawed only once prior to analysis.

Enzyme-Linked Immunosorbent Assays (ELISA)

Two specific proteins were initially investigated as potential serum biomarkers of ectopic pregnancy: vascular endothelial growth factor (VEGF) and pregnancy-associated plasma protein A (PAPP-A). Both proteins were assayed by ELISA as recommended by the test kit manufacturers (Pierce-Endogen, Rockford, Ill. and Genemed Biotechnologies, South San Francisco, Calif., respectively).

Surface-Enhanced Laser Desorption/Ionization (SELDI)

Proteomic profiling of serum proteins was performed using Ciphergen's Protein-Chip System for SELDI, an affinity-based mass spectrometric method in which proteins of interest are selectively adsorbed to a chemically modified surface on a biochip. Various chip chemistries (hydrophobic, ionic, cationic, and metal-binding) were initially evaluated to determine which affinity chemistry provided the best serum profiles in terms of number and resolution of proteins. The weak cation exchange (WCX2) and IMAC-Cu metal binding chip arrays were found to give the best results. The WCX2 findings are presented here.

30 μl of dissolution buffer (8 mol/L urea and 10 g/L CHAPS in phosphate-buffered saline solution, pH 7.2) was added to 20 μL of each serum sample. The mixture was vortex-mixed at room temperature for 15 minutes and further diluted 1:8 in 100 mM sodium acetate buffer (pH 4.0). 100 μL of the diluted samples were applied to each spot on a WCX2 ProteinChip Array using a 96-well Bioprocessor (Ciphergen Bio-systems, Inc., Fremont, Calif.), a device that holds 12 chips and allows application of larger volumes of serum to each chip array. After the samples were incubated at room temperature for 60 minutes on a platform shaker, the array was washed three times with 100 μL of 100 mM sodium acetate buffer (pH 4.0) for 5 minutes, followed by two rinses with 100 μL of distilled water. After air drying, 0.5 μL of saturated sinapinic acid prepared in 500 mL/L acetonitrile-5 mL/L, trifluoroacetic acid was applied twice to each spot.

Serum proteins bound to the WCX2 array were detected with the ProteinChip Reader (Ciphergen Biosystems, Inc.). Data were collected by averaging 91 laser shots with a laser intensity of 240 and a detector sensitivity of 6, molecular mass range 2,000-20,000 Da (Da), and a focus lag time of 746 nanoseconds (ns). Reproducibility was estimated using one representative serum sample. The coefficient of variation was estimated for the selected mass peaks. Mass accuracy was calibrated externally using molecular mass standard containing three peptides, achieving a mass accuracy of 0.1%.

Preliminary analysis of the data was performed with the Ciphergen Biomarker Wizard Software. Peaks detected by the system were then more extensively evaluated using the Classification and Regression Tree (CART) software package of Salford Systems (San Diego, Calif.). CART® is a computationally intensive program that works by recursive partitioning of the sample based upon marker values (one at a time, then in combination) and maximizing a function of the sensitivity and specificity of the sample.

### Results

Specific Marker Analysis

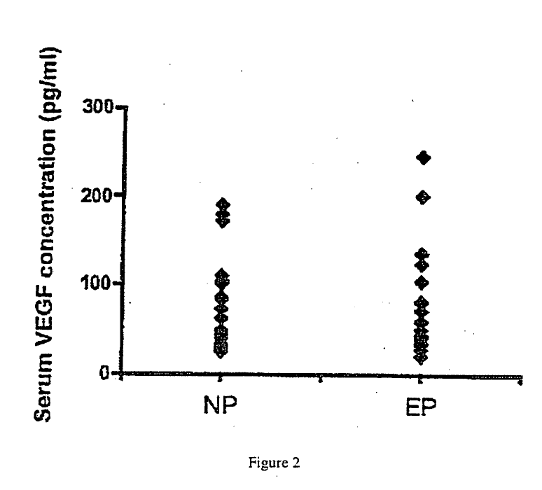

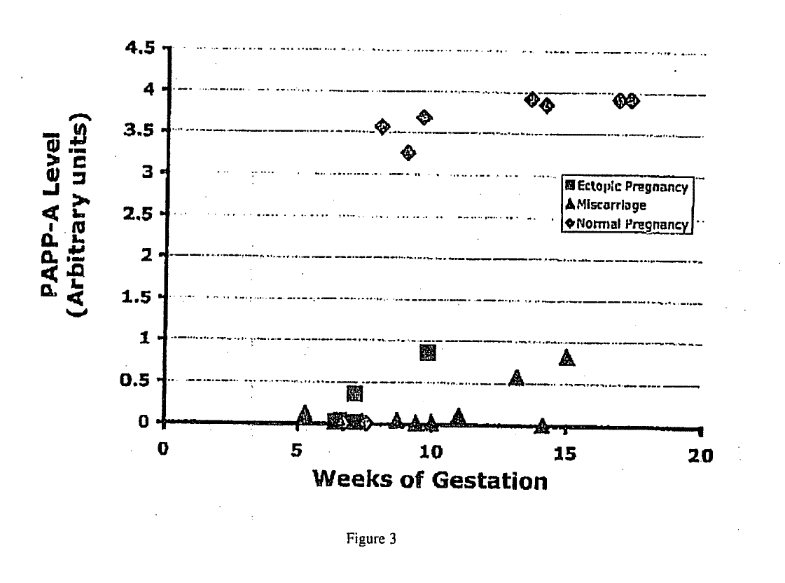

Oviduct-specific glycoprotein in maternal serum was first unsuccessfully tested as an ectopic pregnancy marker (detection by Western blot). VEGF, which has a role in vasculogenesis, a key feature of maternal-embryonic interactions, was next tested. No detectable differences were observed between levels of VEGF in normal versus ectopic pregnancy (FIG. 2). Pregnancy-associated plasma protein A (PAPP-A) was next tested. PAPP-A levels rose dramatically at around 8 weeks of gestation in women with normal pregnancies; otherwise, levels were essentially baseline in women with both normal and ectopic pregnancies (FIG. 3). In general, this method is unlikely to find widespread application, because most patients with ectopic pregnancy have gestational ages of less than 8 weeks at presentation.

Screening for Additional Markers Using SELDI

Next, SELDI, an unbiased method, was utilized to identify additional biomarkers for ectopic pregnancy. SELDI was chosen over other methods, such as two-dimensional gel electrophoresis, because mass spectrophotometry is highly accurate and sensitive, multiple platforms are available (ion exchange [cation and anion], hydrophobic, metal affinity, hydrophobic), and small molecular mass (<10,000 Da) analytes can be detected. An additional major benefit is that all data are collected by computer, enabling facile analysis of multiple variables.

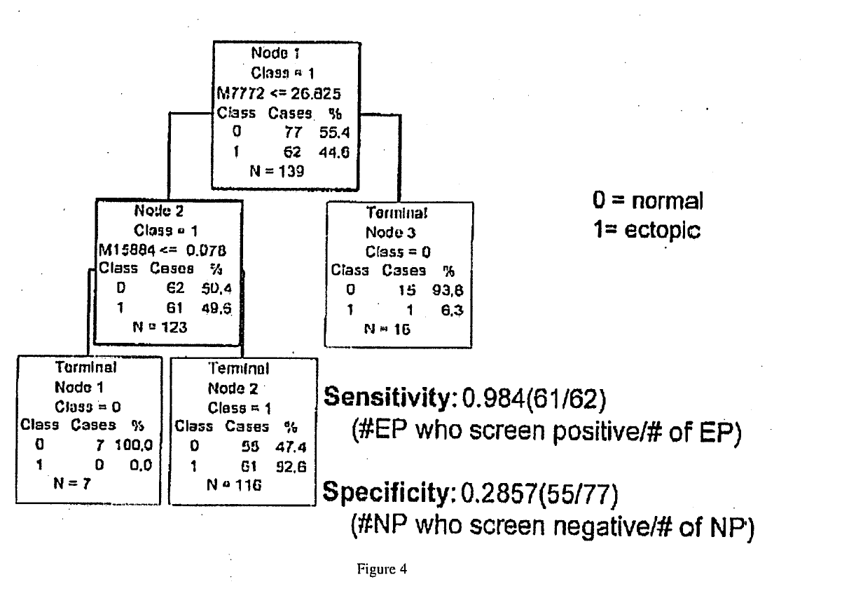

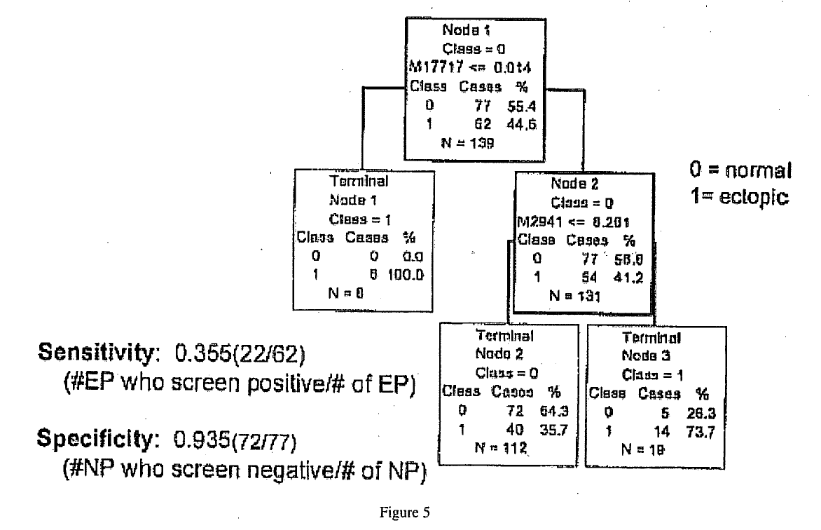

Using this method, molecules in the 2,000-20,000 Da range were analyzed as potential ectopic pregnancy markers. A large number of proteins were identified as potential markers, none of which alone served as a good marker for ectopic pregnancy. Using CART, several combinations proteins were found to have predictive value. However, when considering false positive and false negative errors to have equal cost, the sensitivity (number of EP who screen positive/number of EP) and specificity (number of normal pregnancies who screen negative/number of normal pregnancies) for these analyses were still insufficient. Therefore, the parameters for the CART analysis were modified so as to focus separately on sensitivity and specificity. The following criteria were used to select useful peaks: (1) either 90% sensitivity with a minimum of 20% specificity or 90% specificity with a minimum of 20% sensitivity; and (2) a signal to noise ratio of greater than 3 and a minimum absolute value. These criteria enabled optimization of sensitivity (98.4%) with one set of proteins (masses of 7772 Da and 15,884 Da), and of specificity (93.5%) with two other proteins (masses of 17,717 Da and 2,941 Da). FIGS. 4 and 5 summarize each of the two-node pathways derived by this analysis.

The two trees were next combined into a single paradigm (Table 2), enabling segregation of patients into three groups: those with a high likelihood of having a normal pregnancy (“send home”), those likely to have an ectopic pregnancy (“intervene”), and those in whom an ectopic pregnancy is uncertain and who should be followed closely until a definitive diagnosis can be made (“monitor”). By these criteria, 73 of our patients were identified as normal, 1 of whom actually had an ectopic pregnancy (95.7% correct diagnosis). Most patients (89 of 139) were placed in the “monitor” category; of these, 43.8% had an ectopic pregnancy. Of the 27 patients identified for intervention, 22 of 27 had an ectopic pregnancy (81.5% correct diagnosis).

TABLE 2
Resulting outcomes based on a paradigm that takes into account the
two previous decision trees. Cases expressed as number (Frequency)
and percent (Row pet) are read across each row. Distribution of the
patients with normal pregnancies (Normal) and ectopic pregnancies
(Ectopic) are read down the columns by number (Frequency)
and percent (Col pet).

| | Outcome | | |
|---|---|---|---|
| Predicted | Normal | Ectopic | Total |
| Send home | | | |
| Frequency | 22 | 1 | 23 |
| Row pct | 95.65 | 4.35 | |
| Col pct | 28.57 | 1.61 | |
| Monitor | | | |
| Frequency | 50 | 39 | 89 |
| Row pct | 56.18 | 43.82 | |
| Col pet | 64.94 | 62.9 | |
| Intervene | | | |
| Frequency | 5 | 22 | 27 |
| Row pct | 18.52 | 81.48 | |
| Col pct | 6.49 | 35.48 | |
| Total | 77 | 62 | 139 |

### Example 2

### Further Improvement of the Ectopic Pregnancy Test

An important measure of a proposed diagnostic test is its reproducibility and validity in an independent sample. Thus, the 4-protein diagnostic test of Example 1 was tested in an independent sample. This test sample of 16 EP and 18 normal pregnancy subjects demonstrated that three of our potential peaks were reproducible, while the peak of 17717 Da was less reproducible. This peak was eliminated from future analyses and replaced with a different peak with a molecular mass of about 3962 Da. The 3962 Da peak was used in the same type of decision tree as the 17717 Da peak; i.e, a combination of a high level of the 3962 Da peak and a low level of the 2941 Da peak predicted a non-ectopic pregnancy. This assay predicted ectopic pregnancies with high specificity, as detailed below.

Using these four peaks, a diagnostic decision algorithm was developed. The algorithm sorted patients into three categories. The first category was a subset of patients with a very high likelihood of having a normal pregnancy, which may be sent home with no additional tests. The second category was a subset with a very high likelihood of having an ectopic pregnancy, which should be administered dilation and curettage or laparoscopy to distinguish an ectopic pregnancy from a miscarriage. The third category was those patients which could not be classified into either group with high probability, and which should be followed and diagnosed using current standard tests (serial blood tests, ultrasound or surgical intervention). This algorithm was designed to minimize misdiagnoses and the possibilities of interrupting a normal pregnancy or failing to detect an ectopic pregnancy. This algorithm was performed on the samples from the subjects in Examples 1 and 2, with the following results:

TABLE 2
Sensitivity and specificity of improved algorithm.

| | Original test sample | | |
|---|---|---|---|
| | (Example 1 | Mathematical | Validation sample |
| | subjects) | validation | (Example 2 subjects) |
| Sensitivity | 98.4% | 87.1% | 62.5% |
| Specificity | 97.4% | 79.2% | 100% |

### Example 3

### Identification of the 2941 Da Protein and Another Peptide with Possible Diagnostic Value

### Methods

Protein identification was performed by peptide fragmentation, using a tandem mass spectrometer equipped with a PCI-1000 ProteinChip® (Ciphergen) Interface. Single MS and MS/MS spectra were acquired on a tandem mass spectrometer, either a Q-Star® (ABI) or Q-TOF® (Micromass) equipped with a PCI-1000 ProteinChip Interface. Using ProteinChip Arrays as supplied, without further addition of CHCA (α-ciano-4-hydroxycinnamic acid), spectra were collected in the 1-3 kDa range in single MS mode. After reviewing the spectra, specific ions were selected and introduced into the collision cell for CID fragmentation. The CID spectral data was submitted to the database-mining tool Mascot (Matrix Sciences), a search engine that uses mass spectrometry data to identify proteins from primary sequence databases.®

### Results

Direct sequencing of the 2941 Da protein revealed it to be a peptide fragment from the C-terminal region of the α-fibrin protein of 2931 Da theoretical mass. An additional peptide with possible diagnostic value was discovered as well, a fragment of human serum albumin with a theoretical mass of 2866.5 Da (2871 by SELDI). The sequences of the two peptides are depicted in Table 6 below.

TABLE 6
Sequences of two diagnostic peptides

| | |
|---|---|
| 2941 Da peptide | SSSYSKQFTSSTSYNRGDSTFESKSY (SEQ ID No.1) |
| 2866 Da peptide | DAHKSEVAHRFKDLGEENFKALVLI (SEQ ID No. 2) |

### Example 4

### Identification of 7772 Da, 15884 Da, 3962 Da, and 17717 Da Ectopic Pregnancy Markers

The protocol described in Example 3 is used to identify the 7772 Da, 15884 Da, 3962 Da, and 17717 Da ectopic pregnancy markers described in Example 1-2. Identification of these markers enables the use of additional tests such as any of various known immuno-assays, to assess their concentrations.

### Example 5

### Identification of Additional Ectopic Pregnancy Markers

SELDI, mass spectrometry, and CART analysis are used to identify additional ectopic pregnancy markers as described in Examples 1-2. The additional markers are used to further enhance the sensitivity and specificity of the diagnostic algorithms of the present invention.

### Example 6

### Multivariate Statistical Analysis of Ectopic Pregnancy Markers

CART analysis is used to analyze the marker proteins identified in Examples 1-5 in combination with maternal age, gestational age, clinical history, serum hCG level, and/or other known factors, to improve their accuracy in predicting ectopic pregnancy.

### Example 7

### Use of Multivariate Statistical Analysis to Identify Additional Ectopic Pregnancy Markers

CART analysis is performed on candidate marker protein concentrations in combination with maternal age, gestational age, clinical history, serum hCG level, and/or other known factors. This analysis will identify marker proteins that are diagnostic of ectopic pregnancy when considered in combination with the additional factor(s), even though the concentration of the marker protein, when considered alone, may have less or no diagnostic value.

### Example 8

### Identification of Markers of Miscarriage

The methods described in Examples 1-7 are used to identify marker proteins that distinguish miscarriages from normal pregnancies, and to develop algorithms to determine the risk of a particular patient having a miscarriage.

### Example 9

### Identification of Markers of Hydatiform Pregnancies

The methods described in Examples 1-7 are used to identify marker proteins that distinguish hydatiform (mole) pregnancies from normal pregnancies, and to develop algorithms to determine the risk of a particular patient having a hydatiform pregnancy.

The foregoing has been a description of certain non-limiting embodiments of the invention. Those of ordinary skill in the art will appreciate that various changes and modifications to this description may be made without departing from the spirit or scope of the present invention, as defined in the following claims.

What is claimed is:

1. A method of diagnosing or predicting the existence of ectopic pregnancy in a subject, comprising the steps of:
a. determining an amount of a first and a second marker in a biological sample of said subject, said first marker being a protein or a peptide having a molecular mass of about 7772 daltons and said second marker being a protein or a peptide having a molecular mass of about 15884 daltons; and
b. comparing the amount of said first and second marker to a reference standard for said first and second marker respectively, whereby if the amount determined for either the first or the second standard falls within the range defined by the reference standard for said first or second marker, then the subject is at a low risk of having ectopic pregnancy.

2. The method of claim 1, whereby the step of determining the amount of said first or second marker protein or peptide comprises an immunological assay, a surface-enhanced laser desorption/ionization (SELDI) assay, a mass spectrometry, or a combination thereof.

3. The method of claim 1, wherein said biological sample is a serum sample.

4. A method of diagnosing or predicting of the existence of ectopic pregnancy in a subject, comprising the steps of:
a. determining an amount of a first and a second marker in a biological sample of said subject, said first marker being a protein or a peptide having a molecular mass of about 3962 daltons and said second marker being a protein or a peptide; and
b. comparing the amount of said first and second marker to a reference standard for said first and second marker respectively, whereby if the amount determined for the first marker falls below the range specified for that marker by the standard for said first marker or the second standard falls outside the range defined by the reference standard for said second marker, or both, the subject is at a high risk of having ectopic pregnancy.

5. The method of claim 4, wherein said second marker protein or peptide is a fragment of an alpha-fibrin protein.

6. The method of claim 4, wherein said fragment of an alpha-fibrin protein or peptide has a molecular mass of about 2941.

7. The method of claim 5, wherein said fragment is represented by the sequence set forth in SEQ ID No. 1.

8. The method of claim 4, whereby the step of determining the amount of said first or second marker protein or peptide comprises an immunological assay, a surface-enhanced laser desorption/ionization (SELDI) assay, a mass spectrometry, or a combination thereof

9. The method of claim 4, wherein said biological sample is a serum sample.

10. A method of diagnosing or predicting of the existence of ectopic pregnancy in a subject, comprising the steps of:
a. determining an amount of a first and a second marker in a biological sample of said subject, said first marker being of an alpha-fibrin protein of a biological sample of said subject having a molecular mass of about 2931 Daltons and said second marker being a protein or a peptide having a molecular mass of 3962; and
b. comparing the amount of said first and second marker to a reference standard for said first and second marker respectively, whereby if the amount determined for the alpha-fibrin protein of the biological sample exceeds the range specified for the alpha-fibrin protein of a biological sample by the standard for said alpha-fibrin protein of a biological sample, or the second standard falls outside the range defined by the reference standard for said second marker, or both, the subject is at a low risk of having ectopic pregnancy.

11. The method of claim 10, wherein said fragment of an alpha-fibrin protein is represented by the sequence set forth in SEQ ID No. 1.

12. The method of claim 10, whereby the step of determining the amount of said first or second marker protein or peptide comprises an immunological assay, a surface-enhanced laser desorption/ionization (SELDI) assay, a mass spectrometry, or a combination thereof.

13. The method of claim 10, wherein said biological sample is a serum sample.

14. A method of diagnosing or predicting of the existence of ectopic pregnancy in a subject, comprising the steps of:
a. determining an amount of a first, a second, a third and a fourth marker in a biological sample of said subject, said first marker being a protein or peptide having a molecular mass of about 7772 Daltons; and said third marker being a protein or a peptide having a molecular mass of 3962; and
b. comparing the amount of said first and second marker to a reference standard for said first, second, third and fourth marker respectively, whereby if the amount determined for the first, second, third and fourth marker falls within the range specified for the first, second, third and fourth marker by the standard for said first, second, third and fourth markers respectively, the subject is at a low risk of having ectopic pregnancy.

15. The method of claim 14, wherein if the amount determined for the third marker falls below the range specified for that marker by the standard for said third marker or the fourth marker falls outside the range defined by the reference standard for said fourth marker, or both, then the subject is at a high risk of having ectopic pregnancy.

16. The method of claims 14 or 15, wherein said second marker protein or peptide has a molecular mass of about 15884 Daltons.

17. The method of claims 14 or 15, wherein said fourth marker protein or peptide is a fragment of an alpha-fibrin protein.

18. The method of claim 17, wherein said fragment of an alpha-fibrin protein or peptide has a molecular mass of about 2931.

19. The method of claim 18, wherein said fragment comprises a peptide represented by the sequence set forth in SEQ ID No. 1.

20. The method of claim 14, whereby the step of determining the amount of said first, second, third or fourth marker or their combination comprises an immunological assay, a surface-enhanced laser desorption/ionization (SELDI) assay, a mass spectrometry, or a combination thereof

21. The method of claim 14, wherein said biological sample is a serum sample.

22. The method of claim 1, further comprising determining the amount of a third and a fourth marker, said third and fourth marker being a protein or a peptide having a molecular mass of 3962 Daltons and 2931 Daltons respectively, wherein if said amount of the third marker falls lower than the range defined by the reference standard for said third marker; said amount of the fourth marker falls outside a range defined by said reference standard for said fourth marker protein or peptide, or both, then the subject is at a high risk for developing ectopic pregnancy.

23. The method of claim 22, wherein said fourth marker is a fragment of an alpha-fibrin protein.

24. The method of claim 23, wherein said fragment is represented by the sequence set forth in SEQ ID No. 1.

25. The method of claim 22, whereby the step of determining the amount of said third or fourth marker or their combination comprises an immunological assay, a surface-enhanced laser desorption/ionization (SELDI) assay, a mass spectrometry, or a combination thereof.

26. The method of claim 22, wherein said biological sample is a serum sample.

27. A method of screening for a candidate compound having a therapeutic activity for preventing ectopic pregnancy, comprising the steps of:
a. evaluating a probability of the existence of ectopic pregnancy in a subject according to the method of any one of claims 1, 4, 10 or 14;
b. contacting said subject with said candidate compound; and
c. re-evaluating the probability of the existence of ectopic pregnancy in the subject according to the method of said step of evaluating, wherein finding a high probability of the subject having ectopic pregnancy in the initial evaluation and subsequent change in the probability of the subject having ectopic pregnancy following the reevaluation indicates that the compound has therapeutic activity for preventing ectopic pregnancy.

28. The method of claim 27, comprising a second subject exhibiting same probability of having ectopic pregnancy, wherein the second subject is not contacted with the candidate compound and whose probability of having ectopic pregnancy has not changed upon the step of reevaluating.

29. A method for sorting a subject based on the subject being at risk of having ectopic pregnancy, comprising the steps of:
a obtaining a biological sample from the subject;
b. analyzing the subject's biological sample for the amount of a first marker being a protein or a peptide having a molecular mass of about 7,772 Da, a second marker being a protein or a peptide having a molecular mass of about 15,884 Da, a third marker being a protein or a peptide having a molecular mass of about 17,717 Da and a fourth marker being a protein or a peptide having a molecular mass of about 2,941 Da.;
c. comparing the amount of the first, second, third and fourth markers to a standard corresponding specifically to said first, second, third and fourth markers; and
d. sorting the subjects based on the amount of the first, second, third and fourth markers relative to the range defined by the standards specific for the first, second, third and fourth markers, whereby if:
(i) the first marker falls below the threshold defined by the standard for the first marker, and the second marker falls below the threshold specified for the second marker and the third marker exceeds the threshold specified for the third marker and the fourth marker exceeds the threshold specified for the fourth marker, the subject is at a low risk of having ectopic pregnancy and is assigned a “send home” sorting criteria; or
(ii) the first marker exceeds the threshold defined by the standard for the first marker, and the second marker exceeds the threshold specified for the second marker and the third marker falls below the threshold specified for the third marker and the fourth marker falls below the threshold specified for the fourth marker, the subject is at a high risk of having ectopic pregnancy and is assigned an “intervene” sorting criteria; or
(iii) the amounts of the first, second, third and fourth marker are in any combination with relation to the threshold specific for the first, second, third and fourth marker, which is not covered by conditions (i) and (ii) above, the subject is assigned a “monitor” criteria, thereby being a method of sorting the subject based on the subject being at risk of having ectopic pregnancy.

30. The method of claim 29, wherein said fourth marker is a fragment of an alpha-fibrin protein.

31. The method of claim 30, wherein said fragment is represented by the sequence set forth in SEQ ID No. 1.

32. The method of claim 29, wherein said biological sample is serum.


SEQUENCE LISTING (2 sequences)

SEQ ID NO: 1 - protein, 26 aa, Human
SSSYSKQFTSSTSYNRGDSTFESKSY

SEQ ID NO: 2 - protein, 25 aa, Human
DAHKSEVAHRFKDLGEENFKALVLI